(19) United States
(12) Patent Application Publication
(10) Pub. No.: US 2019/0235623 A1
(43) Pub. Date: Aug. 1, 2019

(54) Virtual Reality Headset

(71) Applicant: Beacon Centre for the Blind, Wolverhampton (GB)

(72) Inventors: Andrew Pollard, Wolverhampton (GB); John Clarke, Wolverhampton (GB); Arwyn Jones, Wolverhampton (GB); Fernando Loizides, Wolverhampton (GB)

(73) Assignee: Beacon Centre for the Blind, Wolverhampton (GB)

(21) Appl. No.: 16/317,631

(22) PCT Filed: Jul. 12, 2017
(86) PCT No.: PCT/GB2017/052048

§ 371 Date: Jan. 14, 2019

(30) Foreign Application Priority Data
Jul. 12, 2016 (GB) 1612085.9

(51) Int. Cl. G06F 3/01; G02B 7/02; G02B 27/00; G02B 7/09; A61B 3/113

(52) U.S. Cl. CPC G06F 3/013; G02B 7/023; A61B 3/113; G02B 7/09; G02B 27/0093

(57) ABSTRACT

A head-mountable display apparatus comprising a headset (10) configured to fit over a user's eyes, in use, the mask having a rear wall (16) adjacent a user's eyes, in use, and an opposing front wall (12), the apparatus further comprising: at least one display screen (30) for receiving image data and displaying respective images thereon, said display screen (30) being mounted between said front and rear walls (12, 16) of said mask, wherein at least said distal ends of said display screen are curved so as to appear concave when viewed by the user, in use, wherein the rear wall (16) has a pair of apertures (18) therein, through which, in use, a user can view said display screen (30); and at least one optical element (20) mounted between a user's eyes and said display screen (30), when said mask is head mounted for user, wherein the or each optical element (20) has an adjustable optical property.

This invention relates generally to a virtual, actual or augmented reality headset and, more particularly but not necessarily exclusively, to a virtual or augmented reality headset adapted for use by visually impaired users, especially (although again not necessarily exclusively) those having central vision sight loss.

Sight loss in the UK—i.e. that which is not correctable by refraction and use of spectacle or contact lenses—already affects some two (2) million people in the UK. This number will double by 2050 largely due to an aging population and consequential symptoms from increasing conditions such as diabetes. The most prevalent sight loss conditions as macular degeneration (central vision loss), glaucoma (peripheral vision loss), diabetic retinopathy (progressive loss of blocks of vision), and cataracts (clouding of the eye lens). Some 750,000 in the UK have central vision sight loss.

Virtual and augmented reality headsets are known and their use is becoming increasingly widespread, especially within the gaming environment. As will be well known to a person skilled in the art, virtual reality is the computer-generated simulation of a three-dimensional image or environment that a user can experience on a screen within a headset and/or interact with in a seemingly real or physical way or, in some cases; whereas actual reality refers to technology that displays live or captured still or moving images of the real world environment on a screen within a headset. Augmented reality is a technology that superimposes a computer generated image on a user's view of the real world, thus providing a composite view. For the avoidance of doubt, any reference herein to either virtual reality (VR) or augmented reality alone is to be considered to encompass all of the above types of technology, unless specifically otherwise stated, and is used herein to refer broadly to any technology for displaying images on a screen within a headset to provide an immersive effect.

A VR headset is typically configured to be mounted for use on a user's head, the headset including a mask configured to fit over a user's eyes, in use, the mask having a rear wall adjacent a user's eyes, in use, and an opposing front wall. A display screen is provided for receiving image data and displaying respective images thereon, the display screen being mounted between the front and rear walls of the mask, wherein the rear wall has a pair of apertures therein, through which, in use, a user can view said display screen. Conventional VR headsets use either two feeds sent to one display screen or two display screens, one per eye. There may also be lenses positioned between a user's eyes and the display screen. In some instances, these can be adjusted horizontally to match the distance between the user's eyes which varies from person to person, and also distance to and from the eyes. These lenses focus and reshape the picture for each eye and create a stereoscopic 3D image by angling the two 2D images to mimic how each of our two eyes views the world slightly differently.

However, conventional VR headsets are designed and configured for use by fully sighted individuals, for use in recreational activities such as gaming. In accordance with aspects of the present invention, it would be desirable to provide a VR headset adapted (and/or adaptable) for use by a partially sighted user, to provide the user with a full (or almost full) 3D immersive experience and, thereby, to improve and enhance their ‘vision’ (especially, but not necessarily limited to, peripheral vision) of the displayed scene(s); and it is an object of embodiments of the present invention to address at least some of these issues.
Thus, in accordance with an aspect of the present invention, there is provided a head-mountable display apparatus comprising a headset configured to fit over a user's eyes, in use, the mask having a rear wall adjacent a user's eyes, in use, and an opposing front wall, the apparatus further comprising:
   at least one display screen for receiving image data and displaying respective images thereon, said display screen being mounted between said front and rear walls of said mask, wherein at least said distal ends of said display screen are curved so as to appear concave when viewed by the user, in use, wherein the rear wall has a pair of apertures therein, through which, in use, a user can view said display screen; and
   at least one optical element mounted between a user's eyes and said display screen, when said mask is head mounted for user, wherein the or each optical element is pivotally adjustable about at least one axis.
In one exemplary embodiment of the present invention the or each optical element may be pivotally adjustable about two or three axes. The invention may optionally comprise means for manual adjustment of said the or each optical element about a first, second or third axis.
Optionally, the apparatus may comprise automatic adjustment means for automatic adjustment of the or each optical element about a first, second or third axis.
In an exemplary embodiment, automatic adjustment means may comprise an eye-tracking device and an optical field testing module, wherein said optical field testing module may be configured to facilitate an optical field test in respect of a user, and said eye-tracking device may be configured to track the user's eye movement during the optical field test, in use, and the apparatus may further comprise a database and a processor for storage and processing of data representative of the user's eye movement.
According to one exemplary embodiment the apparatus may further comprise a data processing module configured to calculate, using data representative of the user's eye movements, automatic adjustment data in respect of the or each said optical elements.
In an exemplary embodiment the optical elements may be removable.
Optionally, at least one of the optical elements may be a focusing lens or a Fresnel lens. Optionally, at least one of the optical elements may be an optical filter. Optionally, the apparatus may afford sufficient space between the rear wall and the user's eyes and around the user's eyes, in use, such that the user can wear the mask over their glasses.
According to one exemplary embodiment a least one of the optical elements is a magnifying or a wide angle lens.
In an exemplary embodiment of the present invention, the apparatus may further comprise two display screens.
Optionally, at least a portion of the or each display screen may be substantially hemi-spherical. At least a portion of the or each display screen may optionally be parabolic.

In accordance with a second aspect of the present invention, there is provided a head-mountable display apparatus comprising a headset configured to be mounted for use on a user's head, the headset including a mask configured to fit over a user's eyes, in use, the mask having a rear wall adjacent a user's eyes, in use, and an opposing front wall, the apparatus further comprising:

at least one display screen for receiving image data and displaying respective images thereon, said display screen being mounted between said front and rear walls of said mask, wherein the rear wall has a pair of apertures therein, through which, in use, a user can view said display screen; and

an optical element mounted between a user's eyes and said display screen, when said mask is head mounted for use, wherein an optical property of said optical element is adjustable, relative to a user's eyes when the mask is mounted for use.

In an exemplary embodiment, one or more optical elements may be mounted between each of said apertures and said display screen. An orientation of the/or each optical element may, optionally, be mechanically adjustable. In this case, the/or each optical element may be manually, or otherwise, pivotally adjustable about two or three axes.

The or at least one of the optical elements may, for example, be a focusing lens, or a magnifying or wide angle lens. The at least one display screen may be removable/replaceable.

An optical element may be mounted within each of said apertures such that an orientation thereof is mechanically adjustable.

The screen may be substantially concave relative to a user's eyes, when mounted for use. In an exemplary embodiment, the screen may be substantially hemispherical.

An optical element may be adjustably mounted within each of said apertures, an orientation of each of said optical elements being independently mechanically adjustable.

In accordance with another aspect of the present invention, there is provided a head-mountable display apparatus comprising a headset configured to be mounted for use on a user's head, the headset including a mask configured to fit over a user's eyes, in use, the mask having a rear wall adjacent a user's eyes, in use, and an opposing front wall, the apparatus further comprising:

a display screen for receiving image data and displaying respective images thereon, said display screen being mounted between said front and rear walls of said mask, wherein the rear wall has a pair of apertures therein, through which, in use, a user can view said display screen; and

an optical element removably mounted within each of said apertures.

Once again, the screen may be substantially concave, e.g. hemispherical, relative to a user's eyes, when the mask is mounted for use.

An orientation of each of said optical elements may be independently mechanically adjustable. In this case, each optical element may be manually, or otherwise, pivotally adjustable about two or three axes.

At least one of the optical elements may, for example, be a focusing lens, or a magnifying or wide angle lens.

In accordance with yet another aspect of the present invention, there is provided a head-mountable display apparatus comprising a headset configured to be mounted for use on a user's head, the headset including a mask configured to fit over a user's eyes, in use, the mask having a rear wall adjacent a user's eyes, in use, and an opposing front wall, the apparatus further comprising:

a display screen for receiving image data and displaying respective images thereon, said display screen being mounted between said front and rear walls of said mask, wherein the rear wall has a pair of apertures therein, through which, in use, a user can view said display screen, wherein said display screen is substantially concave relative to a user's eyes, when mounted for use; and

an optical element mounted between a user's eyes and said display screen, when said mask is head mounted for use.

The display screen may be substantially hemispherical or other curved shape.

An optical property of said optical element may be adjustable relative to a user's eyes when the mask is mounted for use. In this case, an optical element may be mounted between each of said apertures and said display screen.

In an exemplary embodiment, an orientation of the/or each optical element may be mechanically adjustable. In this case, the/or each optical element may be manually, or otherwise, pivotally adjustable about two or three axes.

The or at least one of the optical elements may, for example, be a focusing lens, or a magnifying or wide angle lens.

An optical element may be mounted within each of said apertures such that an orientation thereof is mechanically adjustable. An optical element may be removably mounted within each of said apertures. In this case, an orientation of each of said optical elements may be independently mechanically adjustable. Indeed, each optical element may be manually, or otherwise, pivotally adjustable about two or three axes.

The apparatus may further comprise at least one optical imaging device for generating said image data. The optical imaging device may, for example, comprise a camera, such as a video camera. Alternatively, the optical imaging device may comprise a reflective lens or mirror. The apparatus may comprise at least two optical imaging devices for generating stereoscopic image data. In other words, the apparatus may comprise at least two cameras, or at least two reflective lenses or mirrors.

The at least one optical imaging device may be mounted on said headset. The at least one optical imaging device may be removably mounted on said headset.

The apparatus may further comprise a battery pack and/or a Global Positioning System module mounted on, or integrally housed within, said headset.

The apparatus may further comprise a wireless communication module configured to wirelessly receive said image data from a remote location, said wireless communication module being mounted on, or integrally housed within, said headset.

The peripheral edges of the mask at an open end thereof opposite said front wall may be configured to abut a user's face, in use, so as to limit or block light entering an enclosure defined by the mask. The peripheral edges of the open end of the mask abutting a user's face, in use, may be provided with a resiliently compressible sealing layer of, for example, memory foam or rubber.

The peripheral edges of each of said apertures may be provided with a resiliently compressible sealing layer of, for example, memory foam or rubber.

A distance of said display screen from said front wall may be adjustable. The apparatus may comprise at least two display screens mounted side-by side between said front and rear walls of said mask.

In accordance with yet another aspect of the present invention, there is provided a method of manufacturing a head-mountable display apparatus, comprising the steps of:

providing a headset including a mask configured to fit over a user's eyes, in use, the mask having a rear wall adjacent a user's eyes, in use, and an opposing front wall, and wherein said rear wall has a pair of apertures therein;

mounting, or otherwise providing, between said front and rear walls of said mask, a display screen for receiving image data and displaying respective images thereon; and

selecting one or more optical properties for directing a light path from said screen to a specified portion of each said aperture; and either:

mounting, relative to each of said apertures, an optical element having said respective selected one or more optical properties; or

adjusting an optical element provided in respect of each said aperture so as to have said respective selected one or more optical properties.

These and other aspects of the invention will be apparent to a person skilled in the art from the following specific description, in which embodiments of the invention are described, by way of examples only, and with reference to the accompanying drawings, in which:

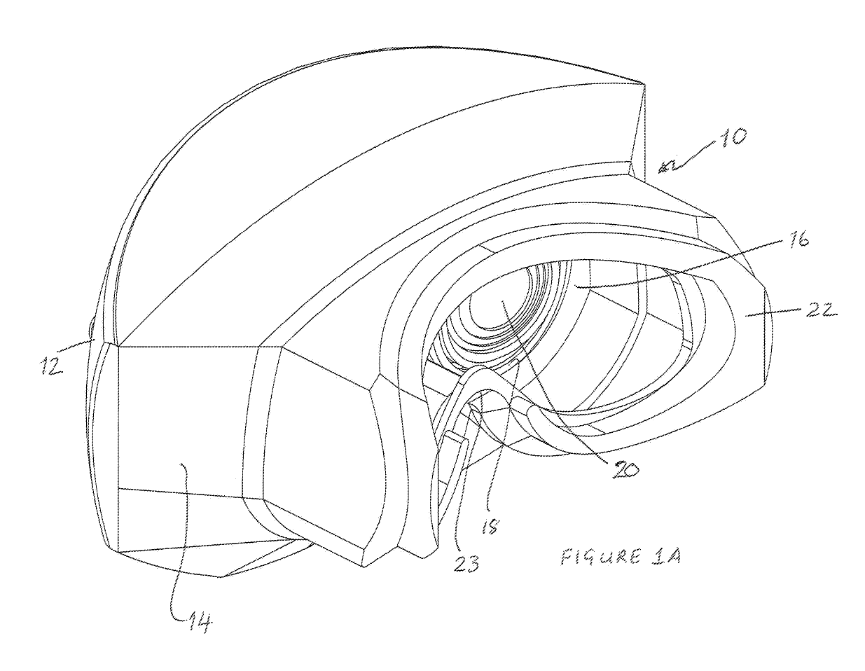

FIG. 1A is a schematic perspective view of a head-mountable display apparatus according to an exemplary embodiment of the present invention;

FIG. 1B is a schematic rear view of a mask of the head-mountable display apparatus of FIG. 1;

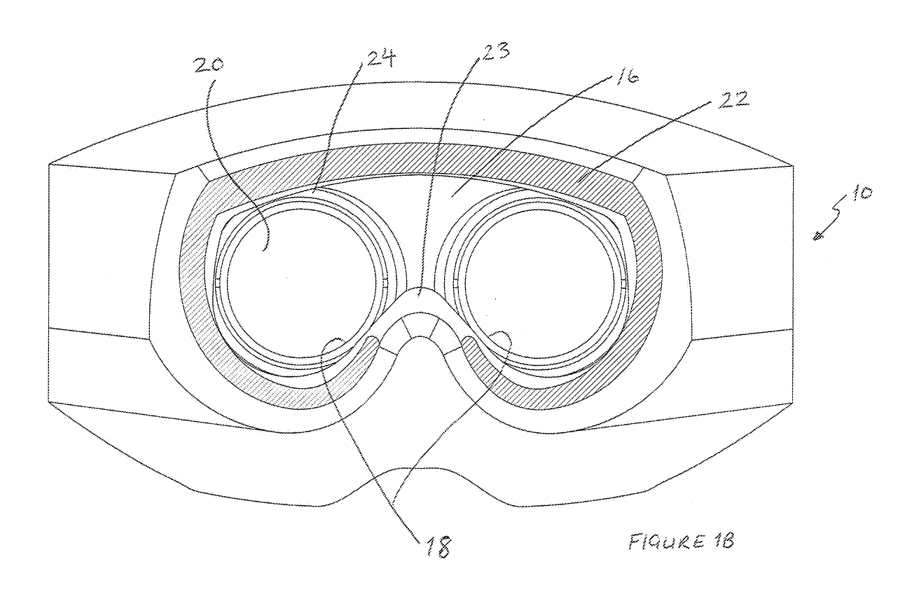

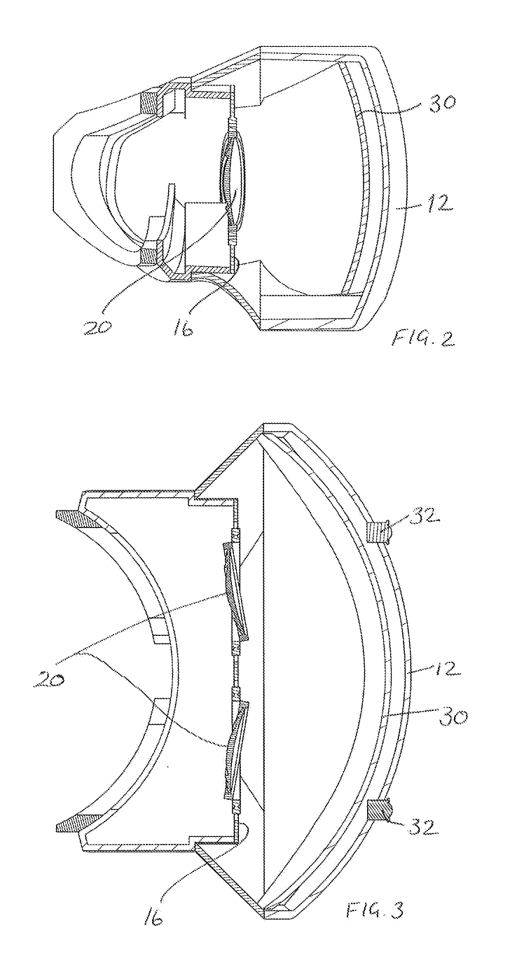

FIG. 2 is a schematic side cross-sectional view of the mask of FIGS. 1A and 1B;

FIG. 3 is a schematic plan cross-sectional view of the mask of FIGS. 1A ands 1B;

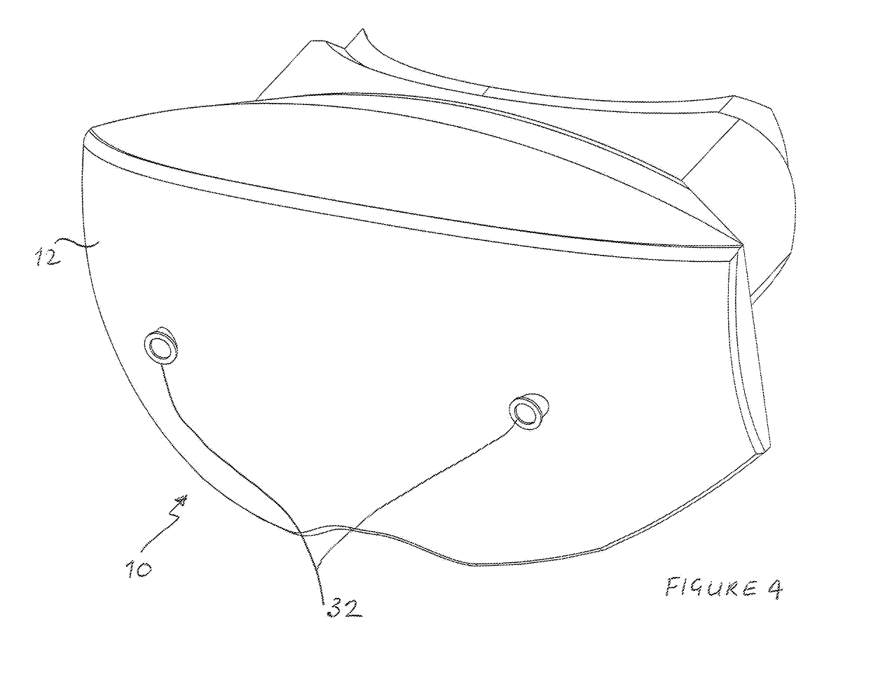

FIG. 4 is a schematic perspective front view of a head-mountable display apparatus according to another exemplary embodiment of the present invention;

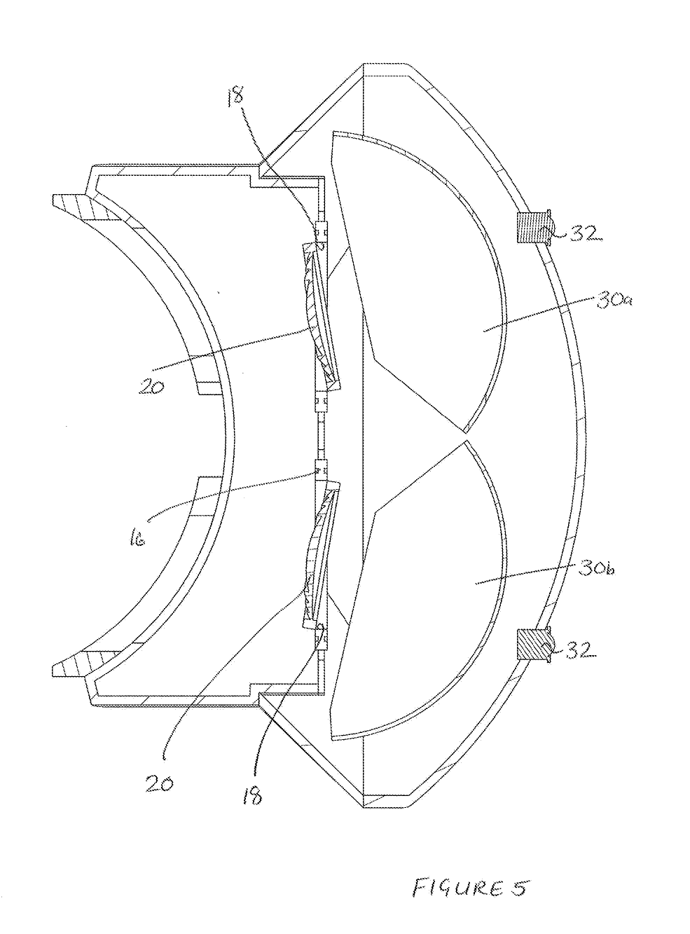

FIG. 5 is a schematic plan cross-sectional view of a head-mountable display apparatus according to an exemplary embodiment of the present invention;

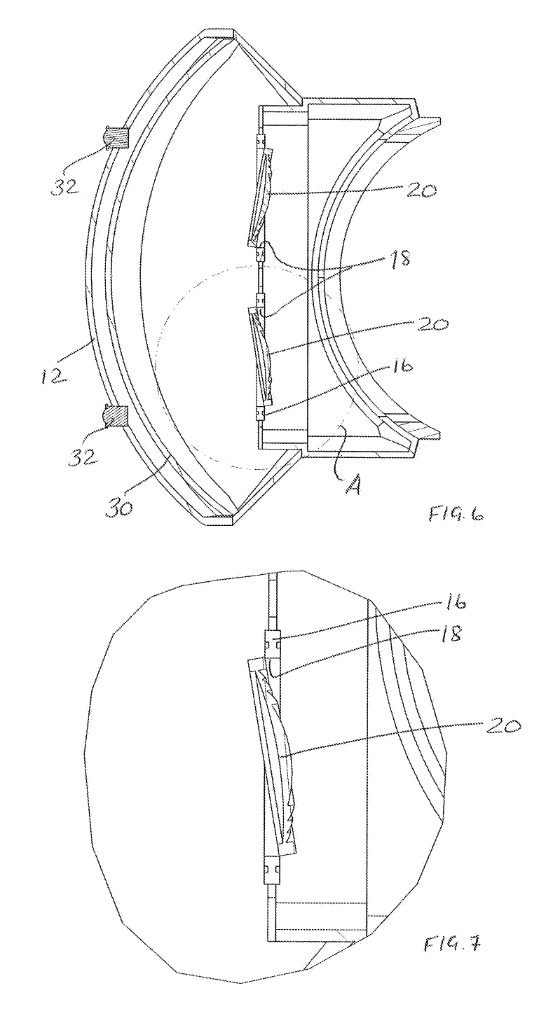

FIG. 6 is a schematic plan cross-sectional view of a head-mountable display apparatus according to an exemplary embodiment of the present invention; and

FIG. 7 is a schematic close-up view of the portion denoted A in FIG. 6.

In general, an object of exemplary embodiments of the invention is to provide apparatus that enables the stereoscopic projection, in close proximity to a user's eyes, of live or recorded high definition, moving or stationary images, such that clinical gaps in the user's vision are either reduced or eliminated. Additionally, the present invention provides means to be user-adaptable based on that user's specific needs, in particular, for a user suffering from conditions that predominantly give rise to central vision loss the lenses and display can be altered in order to project the images onto a different part of the user's retina in order to support their peripheral vision. Therefore the invention provides a fully (or almost fully) 3D immersive effect and, more generally, provides the user with a much improved overall quality of sight. Referring to FIGS. 1A, 1B, 2 and 3 of the drawings, a head-mountable display apparatus according to an exemplary embodiment of the present invention comprises a mask 10 in the form of a housing having a front wall 12 and peripheral side walls 14. The housing is generally open at the end opposite the front wall 12, wherein, in use, the open end of the housing, which is generally rectangular with rounded edges in this exemplary embodiment, is placed or affixed over a user's eyes. A rear wall 16 is provided between the open end and the front wall 12. The rear wall 16 has a pair of apertures 18 defined therein, which are in side-by-side spaced apart relation and at locations that generally correspond to the location of a user's eyes when the mask is placed or affixed thereover for use. Each aperture 18 has mounted therein an optical element 20, which will be described in further detail hereinafter.

The peripheral edges of the open end of the housing are provided with a layer 22 of cushioning material, such as memory foam, rubber or the like, which serves two purposes: not only does it enhance the comfort of the user when wearing the mask for prolonged periods of time, it also acts to seal the join between the open end of the mask and the user's face so as to limit or prevent ambient light from entering the viewing space within the mask. This is particularly important in user's with macular degeneration as it is their peripheral vision which is mostly used, and therefore light coming in from the sides, top and bottom of the device can interfere with the quality of the experience.

As shown in FIGS. 1A and 1B of the drawings, the cushioning material 22 may be provided around most of the periphery of the open end of the housing, leaving a small gap at the lower edge (when the mask is oriented for use) corresponding to the location at which the bridge of a user's nose would be, in use. Here, a small recess or indentation 23 may be defined to accommodate the bridge of a user's nose, for added comfort and security.

It can be seen from FIGS. 2 and 3 of the drawings, that a display screen 30 is mounted within the mask housing, at or adjacent to the front wall 12. The display screen 30, in this exemplary embodiment of the invention, is substantially rectangular in plan, such that the screen 30 extends across the eyes of the user. The display screen 30 comprises, at least at its ends, a segment of a hemisphere, such that it ‘wraps around’ the users field of vision, in use, and is generally concave from the user's viewpoint within the mask, which enables the invention to provide an optimum wide angled coverage, i.e. a complete field of view. The display screen 30 may generally match the profile of the front wall 12, although the present invention is not necessarily intended to be limited in this regard. Indeed, the front wall may be substantially flat, or have any other profile dictated by design or personal preference considerations. Indeed, and referring to FIG. 5 of the drawings, in an alternative exemplary embodiment of the invention, the apparatus may comprise a pair of display screens 30a, 30b, each positioned opposite a respective aperture/optical element pair. The or each display screen 30 may be substantially hemispherical in shape so as to cover a larger field of view than possible with the use of a flat screen, and provide an all-encompassing imaging space to maximise the imaging space available within the user's peripheral vision. In one exemplary embodiment the edges of the display screen only may be hemi-spherical in shape.

As stated above, and referring additionally to FIGS. 6 and 7 of the drawings, each aperture 18 in the rear wall 16 of the mask is provided with an optical element. In conventional VR systems, these optical elements are fixed lenses that are configured to focus and re-shape the displayed image for each eye to create a stereoscopic 3D image. In other words, the function of the optical elements provided in conventional VR systems is to create the stereoscopic 3D image for fully sighted individuals, and, with the exception of (in some cases) being able to adjust the apertures or lenses horizontally to match the spacing of an individual's eyes, it is a ‘one size fits all’ approach. Such adjustments include translational movement of the lenses in one axis, in order to move the lenses closer or further away from the individual's eyes. Some VR headsets may include the ability to move the lenses along a second axis, so as to allow the lenses to move closer to and further apart from each other, thus enabling the user to align the lenses with their own pupillary distance. In contrast, an object of aspects of the present invention is to provide a system that creates a 3D immersive effect for partially sighted users, whereby the proportion of the displayed images reaching the sighted part of the user's eyes, such as the peripheral region of the retina, is maximised and enhanced. The manner in which this is achieved is dependent on the nature of the user's sight loss and the extent and location of their remaining vision. Thus, in exemplary embodiments of the present invention, the optical elements 20 mounted within the apertures 18 may additionally be configured to direct light from the displayed image specifically toward an area of a user's vision that is unimpaired (or less so) compared with other areas of their vision. Thus, for example, for a user having central vision sight loss, the optical elements 20 would be utilised to direct light from the displayed image to the appropriate peripheral vision area(s) of their eye(s). The brightness, hue, contrast, saturation and/or refresh rate of the screen 30 may be varied so that the respective characteristics of any selected part(s) of the screen (corresponding to a user's field of vision, in use). For example, one or more selected parts of the screen may be made brighter than other parts of the screen. It is important to note that this is not the same as varying the characteristics (e.g. brightness) of selected portions of the image—in that case, the screen would remain of uniform brightness across its visible plane. It is also important to note that this is not the same as the common ability to control the overall characteristics (e.g. brightness) of a display screen which conventionally is applied uniformly over the visible screen area.

The screen brightness (for example) may, of course, vary according to the user and/or the condition of the user's vision. Indeed, in some cases, the user may also have a degree of, for example, myopia that may also require correction to further enhance their 3D immersive experience. Thus, it is envisaged that the optical elements 20 may be removably (and therefore replaceably and/or interchangeably) mounted within the apertures 18. This would allow lenses of differing prescriptions to be mounted within the device. In one exemplary embodiment, for example, the optical elements 20 may be mounted within the apertures 18 by means of a snap-fit, wherein the peripheral edges of the optical elements are snap-fitted into cooperative grooves or channels provided in the inner periphery of the apertures 18, but the present invention is not necessarily intended to be limited in this regard. Thus, the best optical elements to suit a specific user can be selected and mounted within the apertures 18.

Thus, referring back to FIGS. 6 and 7 of the drawings, in this exemplary embodiment, or other exemplary embodiments, of the present invention, the optical elements 20 may be adjustably mounted within the apertures 18, to enable adjustment of their optical properties (relative to the light path between the displayed image and the user's eyes), as required to achieve the above-described object(s). In particular, the optical elements 20 may be pivotally mounted within the apertures 18, such that they may be rotatably adjusted in at least one axis. A common problem with, for example, macular degeneration, is that the area of non-sightedness can change over time. The present invention allows for this by allowing the lenses to be pivotally adjusted and therefore adapted to the user's changing needs. The lenses may be adjusted manually, either by a user or by a specialist technician.

In one exemplary embodiment, it is envisaged that the system further comprises an eye-tracking system, such as those commonly used in studies. An optical field test program could be displayed on the screen 30. During the test, the eye-tracking system would be able to track the user's eye movement and therefore obtain a measurement of the user's optical field. The system would then be able to adjust the lenses according to the results of the test and the adjustment could be done automatically, eliminating the need for the user to go and see a specialist technician or alter the lenses themselves. This has the added advantage of continually improving the user experience, even when they might not notice degeneration in sight quality themselves.

Furthermore, the orientation of the optical elements may be adjusted mechanically in two or three axes to alter their optical properties and direct a maximum proportion of the above-mentioned light path toward a specific area of a user's vision. In yet another exemplary embodiment, the optical elements within the apertures 18 may be of a conventional type, and further optical elements may be adjustably and/or removably (and replaceably) mounted between the rear wall 16 and the display screen 30 to achieve the above-described object(s) for a specific user.

The display screen 30 may also be adjustably mounted within the mask housing such that (at least) the distance between the rear wall 16 and the display screen 30 can be selectively adjusted to suit a user's specific visionary needs. Indeed, and as shown in FIG. 5 of the drawings, in some exemplary embodiments, at least two display screens may be provided at or adjacent to the front wall 12 (each positioned such that the field of vision available to each eye is improved or optimized), in which case, each screen may be independently adjustable in the manner described above.

Referring particularly to FIG. 4 of the drawings, a pair of imaging devices or cameras 32 are mounted externally of the mask on the front wall 12 of the housing, for generating image data to be displayed on the display screen 30. The cameras may, for example, be wide angle or narrower angle cameras, but the present invention is not necessarily limited to any particular type of camera or, indeed, imaging means (for example, reflective lenses or mirrors may alternatively be used to capture the required image data). In this exemplary embodiment, the cameras 32 are mounted at a location substantially matching the location of the user's eye line, such that images captured thereby substantially mimic the user's viewpoint. However, this is not necessarily essential, and the cameras (or other optical elements, such as reflective lenses or mirrors) do not necessarily need to be located within the user's ‘normal’ eye line: instead, image processing techniques, that will be known to a person skilled in the art, can be used to adjust the captured images from a different viewpoint when displayed on the display screen 30. Indeed, the image data may not come from imaging means mounted or otherwise provided on the headset itself. Instead, image data may be received, wirelessly or otherwise, from a remote location.

Image processing for displaying 3D images on a concave or hemispherical screen has been addressed previously in the prior art, and various techniques exist. In some known image processing techniques for this purpose, image stitching may be used to generate fine image and wide angled views. In addition, pre-warping correctional methods may be used to adjust image warping and an alpha-mask method may be used to reduce the intensity of any overlapping areas. However, other methods of processing image data for effective display on a concave or hemispherical screen are known, and the present invention is not necessarily intended to be in any way limited in this regard.

The apparatus may be battery powered, in which case, a battery pack may be mounted on or integrally housed within the headset. Alternatively, or in addition, the apparatus may be mains powered, and a power supply connector may be incorporated. A GPS module (not shown) may also be mounted on or integrally housed in the headset. A strap (not shown) may be provided for enabling a user to affix the mask over their eyes and maintain it there, hands free, for prolonged periods of time.

It will be appreciated that, in accordance with some aspects, the use of a curved, or partially or completely hemispherical, screen may significantly enhance a user's 3D immersive experience, especially if, for example, they have central vision sight loss and the apparatus is required to maximise the proportion of the image data reaching their peripheral vision (on the basis that the screen wraps around at least the edges of the user's field of vision, in use, to provide an optimum wide angled coverage, i.e. a complete field of view. However, in other aspects, the use of the adjustable optical elements and/or adjustable screen elements may be sufficient to meet the required aims. Thus, in this case, the screen (or screens) may be substantially flat, although in some cases, they may be oriented at an angle (relative to the user's viewpoint) and, indeed, in some exemplary embodiments, the orientation of the screen (about the vertical and/or horizontal axes relative to the mask when oriented for use) could be adjustable for this purpose.

It will be apparent to a person skilled in the art, from the foregoing description, that modifications and variations can be made to the described embodiments without departing from the scope of the invention as defined by the appended claims.

What is claimed is:

1. A head-mountable display apparatus comprising a headset configured to fit over a user's eyes, the headset comprising a mask having a rear wall adjacent a user's eyes, and an opposing front wall, the apparatus further comprising:
at least one display screen for receiving image data and displaying respective images thereon, said display screen being mounted between said front and rear walls of said mask, wherein at least said distal ends of said display screen are curved so as to appear concave when viewed by the user, wherein the rear wall has a pair of apertures therein, through which, a user can view said display screen; and
at least one optical element mounted between a user's eyes and said display screen, when said mask is head mounted for user, wherein the or each optical element has an adjustable optical property.

2. Apparatus according to claim 1, wherein the or each optical element is pivotally adjustable about at least one axis.

3. (canceled)

4. Apparatus according to claim 1, further comprising means for manual adjustment of said the or each optical element about a first, second or third axis.

5. Apparatus according to claim 1, further comprising automatic adjustment means for automatic adjustment of the or each optical element about a first, second or third axis.

6. Apparatus according to claim 5, wherein said automatic adjustment means comprises an eye-tracking device and an optical field testing module, wherein said optical field testing module is configured to facilitate an optical field test in respect of a user, and said eye-tracking device is configured to track the user's eye movement during the optical field test, the apparatus further comprising a database and a processor for storage and processing of data representative of the user's eye movement.

7. Apparatus according to claim 6, further comprising a data processing module configured to calculate, using data representative of the user's eye movements, automatic adjustment data in respect of the or each said optical elements.

8. Apparatus according to claim 1, wherein the optical elements are removable.

9.-13. (canceled)

14. Apparatus according to claim 1, comprising a control unit for selectively varying the brightness, hue, contrast, saturation and/or refresh rate of selected portions of the or each display screen.

15. A head-mountable display apparatus comprising a headset configured to be mounted for a user's head, the headset including a mask configured to fit over a user's eyes, the mask having a rear wall adjacent a user's eyes, and an opposing front wall, the apparatus further comprising:
at least one display screen for receiving image data and displaying respective images thereon, said display screen being mounted between said front and rear walls of said mask, wherein the rear wall has a pair of apertures therein, through which, a user can view said display screen; and
an optical element mounted between a user's eyes and said display screen, when said mask is head mounted, wherein an optical property of said optical element is adjustable, relative to a user's eyes when the mask is mounted.

16. (canceled)

17. Apparatus according to claim 15, wherein said at least one display screen is removably mounted.

18. Apparatus according to claim 15, wherein at least one optical element is mounted between each of said apertures and said display screen.

19.-22. (canceled)

23. Apparatus according to claim 15, wherein at least one optical element is mounted within each of said apertures such that an orientation thereof is mechanically adjustable.

24. Apparatus according to claim 15, wherein at least a portion of said screen is substantially concave relative to a user's eyes.

25. (canceled)

26. Apparatus according to claim 15, wherein at least one optical element is adjustably mounted within each of said apertures, an orientation of each of said optical elements being independently mechanically adjustable.

27. Apparatus according to claim 15, comprising a control unit for selectively varying the brightness, hue, contrast, saturation, and/or refresh rate of selected portions of the or each display screen.

28. A head-mountable display apparatus comprising a headset configured to be mounted for a user's head, the headset including a mask configured to fit over a user's eyes, the mask having a rear wall adjacent a user's eyes, and an opposing front wall, the apparatus further comprising:
at least one display screen for receiving image data and displaying respective images thereon, said display screen being mounted between said front and rear walls of said mask, wherein the rear wall has a pair of apertures therein, through which, a user can view said display screen; and
an optical element removably mounted within each of said apertures.

29.-48. (canceled)

49. Apparatus according to claim 28, further comprising at least one optical imaging device for generating said image data.

50.-52. (canceled)

53. Apparatus according to claim 49, comprising at least two optical imaging devices for generating stereoscopic image data.

54.-57. (canceled)

58. Apparatus according to claim 28, further comprising a Global Positioning System module, mounted on, or integrally housed within, said headset.

59. Apparatus according to claim 28, further comprising a wireless communication module configured to wirelessly receive said image data from a remote location, said wireless communication module being mounted on, or integrally housed within, said headset.

60. Apparatus according to claim 28, wherein the peripheral edges of the mask are configured to abut a user's face, so as to limit or block light entering an enclosure defined by the mask.

61.-66. (canceled)

67. Apparatus according to claim 28, wherein the apparatus is mounted within a helmet configured to be worn over a user's head and face.

68. A method of manufacturing a head-mountable display apparatus, comprising the steps of:
providing a headset including a mask configured to fit over a user's eyes, the mask having a rear wall adjacent a user's eyes, and an opposing front wall, and wherein said rear wall has a pair of apertures therein;
mounting, or otherwise providing, between said front and rear walls of said mask, at least one display screen for receiving image data and displaying respective images thereon; and selecting one or more optical properties for directing a light path from said screen to a specified portion of each said aperture; and either:
mounting, relative to each of said apertures, an optical element having said respective selected one or more optical properties; or
adjusting an optical element provided in respect of each said aperture so as to have said respective selected one or more optical properties.
